SEQUENCE LISTING (8 sequences)

SEQ ID NO: 1 - RNA, 64 nt, synthetic construct
  source 1..64: mol_type = other RNA; note = Nucleotide sequence of mir-25802
tcacggatacagcctcctttgggatcctgctctgttcccatgagactgtatctgcctgtgtcca

SEQ ID NO: 2 - RNA, 24 nt, synthetic construct
  source 1..24: mol_type = other RNA; note = miR-25802 mimic
tcacggatacagcctcctttggga

SEQ ID NO: 3 - DNA, 50 bp, synthetic construct
  source 1..50: mol_type = other DNA; note = Reverse transcription primer for the miR-25802
gtcgtatccagtgcagggtccgaggtattcgcactggatacgactcccaa

SEQ ID NO: 4 - DNA, 20 bp, synthetic construct
  source 1..20: mol_type = other DNA; note = Forward primer for qPCR detection of the miR-25802
cgtcacggatacagcctcct

SEQ ID NO: 5 - DNA, 20 bp, synthetic construct
  source 1..20: mol_type = other DNA; note = Reverse primer for qPCR detection of the miR-25802
agtgcagggtccgaggtatt

SEQ ID NO: 6 - RNA, 21 nt, synthetic construct
  source 1..21: mol_type = other RNA; note = Negative control NCM
ttgtactacacaaaagtactg

SEQ ID NO: 7 - RNA, 21 nt, synthetic construct
  source 1..21: mol_type = other RNA; note = Negative control NCI
cagtacttttgtgtagtacaa

SEQ ID NO: 8 - RNA, 24 nt, synthetic construct
  source 1..24: mol_type = other RNA; note = miR-25802 inhibitor
tcccaaaggaggctgtatccgtga

(19) United States
(12) Patent Application Publication
(10) Pub. No.: US 2026/0055461 A1
(43) Pub. Date: Feb. 26, 2026

(54) miR-25802 CLUSTER AS BIOMARKER FOR INFLAMMATION-ASSOCIATED DISEASE, AND USE OF miR-25802 CLUSTER

(71) Applicant: INSTITUTE OF MEDICINAL BIOTECHNOLOGY, CAMS & PUMC, Dongcheng District, Beijing (CN)

(72) Inventors: Rui LIU, Beijing (CN); Zhuorong LI, Beijing (CN); Kaiyue ZHAO, Beijing (CN); Mimin LIU, Beijing (CN); Li ZENG, Beijing (CN)

(21) Appl. No.: 18/713,459

(22) PCT Filed: Jul. 24, 2023
(86) PCT No.: PCT/CN2023/108782

§ 371 Date: May 31, 2024

(30) Foreign Application Priority Data
Dec. 6, 2022 (CN) 202211553371.3

(51) Int. Cl. C12Q 1/6883; C12N 15/113; C12N 15/88

(52) U.S. Cl. CPC C12Q 1/6883; C12N 15/113; C12N 15/88; C12Q 2600/158; C12Q 2600/178

(57) ABSTRACT

The present disclosure provides a miR-25802 cluster as a biomarker for an inflammation-associated disease. The miR-25802 cluster can also serve as a target for treating an inflammation-associated disease.

### CROSS-REFERENCE TO RELATED APPLICATION

This application is a national stage application of International Application No. PCT/CN2023/108782, filed on Jul. 24, 2023 which claims priority to a Chinese Patent Application CN202211553371.3 filed to the China National Intellectual Property Administration (CNIPA) on Dec. 6, 2022 and entitled “miR-25802 CLUSTER AS BIOMARKER FOR INFLAMMATION-ASSOCIATED DISEASE, AND USE OF miR-25802 CLUSTER”, both of which are incorporated herein by references in their entireties.

### REFERENCE TO SEQUENCE LISTING

A computer readable XML file entitled “GWPCTP20231108931”, created on Jan. 24, 2024, with a file size of about 9, 610 bytes, contains the sequence listing for this application, has been filed with this application, and is hereby incorporated by reference in its entirety.

### TECHNICAL FIELD

The present disclosure belongs to the technical field of biological detection, and specifically relates to a miR-25802 cluster as a biomarker for an inflammation-associated disease, and use of the miR-25802 cluster.

### BACKGROUND

Inflammation is a general term for immune responses to endogenous and exogenous stimuli in the body, and is characterized by immune cell activation, increased cytokine and chemokine levels, and increased reactive oxygen species release. As a key pathological mechanism, inflammation widely affects the pathological progression of a variety of chronic diseases, exacerbates the inflammatory pathological damage, and promotes the disease deterioration. Inflammation plays an important role in a variety of chronic diseases such as cancer, cardiovascular diseases, metabolic diseases, and dementias, and in particular, persistent chronic inflammation will cause an irreversible tissue damage and organ dysfunction. A variety of neurodegenerative diseases, such as Parkinson's disease, amyotrophic lateral sclerosis, and Huntington's disease, as well as Alzheimer's disease (AD), have significant inflammatory processes. Currently, anti-inflammatory drugs are classified as steroidal anti-inflammatory drugs and non-steroidal anti-inflammatory drugs. Anti-inflammatory drugs mainly play the roles of antipyretic, analgesic, anti-inflammatory, and anti-rheumatic, and are widely used for the alleviation symptoms of osteoarthritis, rheumatoid arthritis, and a variety of fever and pain clinically. However, the current anti-inflammatory drugs each need to be taken for a long time to exert a long-term anti-inflammatory effect, cannot effectively inhibit the occurrence of inflammation fundamentally, and may cause side effects such as cardiovascular side effects and gastrointestinal side effects. Therefore, there is an urgent need to find an anti-inflammatory target allowing better specificity and more significant therapeutic activity.

AD is a progressive neurodegenerative disease that has an insidious onset and is highly correlated with an age. AD is clinically characterized by cognitive failures such as learning ability decline and memory decline. Major pathological mechanisms of AD are extracellular senile plaque deposition caused by amyloid aggregation and intracellular neurofibrillary tangles caused by tau protein hyperphosphorylation. Due to complicated and unclear pathological mechanisms of AD and lack of reliable biomarkers and effective drug-acting targets for AD, the diagnosis and treatment of AD face severe challenges. Currently, the diagnosis of AD is mainly based on a neuropsychological test accompanied by examination of a pathological marker in a body fluid. This diagnosis method has low sensitivity, poor specificity and accuracy, and poor adaptability. However, the current anti-AD drugs in clinical practice or at a research stage have limited efficacy and cannot delay the progression of AD or cure AD. Therefore, the discovery of reliable biomarkers for AD diagnosis and targets for drug intervention is an urgent scientific problem to be solved in the prevention and treatment of AD.

MicroRNAs (miRNAs) are a class of important endogenous molecules, and the expression of miRNAs is significantly tissue-specific and time-ordered. miRNAs can regulate expression levels of key genes to affect the progression of diseases. Familial AD is closely related to mutations of PSEN1, PSEN2, APP, or the like, and thus the early diagnosis and intervention of familial AD can be conducted through genotyping. However, there is currently no report on related genes for sporadic AD with an incidence rate of 95%. In addition, there is a lack of effective therapeutic targets and reagents for inflammation-associated diseases represented by AD, and the current research on immunoregulation mechanisms of non-coding genes is still at an initial stage. Based on multi-targeting characteristics of miRNAs, a miRNA-mediated epigenetic regulatory mechanism is expected to intervene in an inflammatory process at an upstream gene level by regulating a complicated interactive inflammatory signaling pathway network. Therefore, the discovery of novel genetic biomarkers for AD and the discovery of novel targets for regulating an inflammatory process at a gene level are of great significance for the cure of AD and other chronic diseases caused by inflammation.

### SUMMARY

An objective of the present disclosure is to provide a miR-25802 cluster as a biomarker for an inflammation-associated disease and use of the miR-25802 cluster and to design an early detection kit to effectively diagnose and/or treat an inflammation-associated disease, determine disease outcome, and improve a life quality of a patient.

The present disclosure provides a miR-25802 cluster as a biomarker for an inflammation-associated disease, where the miR-25802 cluster includes any one selected from the group consisting of the following I) to IV):
I) miR-25802, the miR-25802 includes a nucleotide sequence set forth in SEQ ID NO: 2;
II) a derivative obtained by modifying the miR-25802 described in I);
III) a miRNA that has a length of 18 nt to 26 nt and has the same or basically the same function as the miR-25802 described in I); and
IV) a derivative obtained by modifying the miRNA described in III).

Preferably, a precursor of the miR-25802 is mir-25802, and the mir-25802 includes a nucleotide sequence set forth in SEQ ID NO: 1.

The present disclosure also provides use of the miR-25802 cluster described in the above technical solution in one or more selected from the group consisting of the following a1) to a6):
a1) preparation of a kit for screening an inflammation-associated disease population;
a2) preparation of a kit for diagnosing an inflammation-associated disease population;
a3) preparation of a kit for monitoring a therapeutic status of an inflammation-associated disease population;
a4) preparation of a kit for monitoring prognosis of an inflammation-associated disease population;
a5) preparation of a kit for screening targets related to the inflammation-associated disease; and
a6) preparation of a drug for treating the inflammation-associated disease.

Preferably, the inflammation-associated disease includes AD.

Preferably, the drug includes one or more selected from the group consisting of the following b1) to b8):
b1) a drug for promoting activation of a microglial cell;
b2) a drug for promoting a pro-inflammatory cell phenotype of a microglial cell;
b3) a drug for promoting release of a pro-inflammatory cytokine, where the pro-inflammatory cytokine includes TNF-α and/or IL-6;
b4) a drug for inhibiting release of an anti-inflammatory cytokine, where the anti-inflammatory cytokine includes IL-4 and/or IL-10;
b5) a drug for promoting an NF-κB signaling pathway in a microglial cell and promoting a neuroinflammatory response;
b6) a drug for promoting expression of an MI molecular marker in a microglial cell, where the M1 molecular marker in the microglial cell includes iNOS;
b7) a drug for inhibiting expression of an M2 molecular marker in a microglial cell, where the M2 molecular marker in the microglial cell includes ARG1; and
b8) a drug for reducing an expression level of KLF4.

The present disclosure also provides a drug for treating an inflammation-associated disease, where an active ingredient of the drug includes a substance for knocking-out or knocking-down the miR-25802 cluster described in the above technical solution.

Preferably, the active ingredient includes a chemical small-molecule drug, a nucleic acid drug, and an antibody drug.

The present disclosure also provides a primer set for detecting the miR-25802 cluster described in the above technical solution, where the primer set includes a reverse transcription primer, an upstream primer, and a downstream primer; and
the reverse transcription primer includes a nucleotide sequence set forth in SEQ ID NO: 3,
the upstream primer includes a nucleotide sequence set forth in SEQ ID NO: 4, and
the downstream primer includes a nucleotide sequence set forth in SEQ ID NO: 5.

The present disclosure also provides use of the primer set described in the above technical solution in preparation of one or more kits selected from the group consisting of the following c1) to c4):
c1) a kit for screening an inflammation-associated disease population;
c2) a kit for diagnosing an inflammation-associated disease population;
c3) a kit for monitoring a therapeutic status of an inflammation-associated disease population; and
c4) a kit for monitoring prognosis of an inflammation-associated disease population.

The present disclosure also provides a kit for screening an inflammation-associated disease, including the primer set described in the above technical solution.

Preferably, the inflammation-associated disease includes AD.

The miR-25802 cluster provided by the present disclosure includes miR-25802, and the miR-25802 includes a nucleotide sequence set forth in SEQ ID NO: 2. The present disclosure has found through detection of AD model cells, AD model animals, and clinical blood samples that the expression of miRNAs of the miR-25802 cluster in AD is significantly increased, and thus the miR-25802 cluster can be used as a biomarker for detection of AD.

In addition, in the present disclosure, a function of the miR-25802 is subjected to in-depth systematic research through enzyme-linked immunosorbent assay (ELISA), Western Blot, dual-luciferase reporter assay, and gene function gain and knockout assay. It is found that the miR-25802 can induce the activation of a microglial cell and induce a pro-inflammatory cell phenotype, and the down-regulation of expression of the miR-25802 can induce an anti-inflammatory phenotype of a microglial cell. The miR-25802 can positively regulate an NF-κB inflammatory signaling pathway in a microglial cell, induce the activation and phenotype transformation of a microglial cell, and promote an inflammatory response. Therefore, a substance for knocking out or knocking down the miR-25802 can inhibit a microglial cell-mediated innate immune response, improve an inflammatory pathological process of AD, and effectively prevent and treat AD.

### BRIEF DESCRIPTION OF THE DRAWINGS

To explain the technical solutions in the Examples of the present disclosure or in the prior art clearly, the drawings required in the Examples will be briefly described below.

FIG. 1 is a heat map illustrating expression levels of a miR-25802 cluster in cerebral cortices of APP/PSI mice that are determined by miRNA high-throughput sequencing;

FIG. 2A shows expression levels of miR-25802 in an APPswe cell at different time points after a copper ion treatment that are determined by quantitative real-time polymerase chain reaction (qRT-PCR) (an AD nerve cell model);

FIG. 2B shows expression levels of miR-25802 in an LPS/IFN-γ-treated microglial cell that are determined by qRT-PCR (a neuroinflammatory cell model);

FIG. 2C shows expression levels of miR-25802 in APP/PS1 mice and wild-type (WT) control mice that are determined by qRT-PCR (a cortex of an animal model);

FIG. 2D shows expression levels of miR-25802 in APP/PSI mice and WT control mice that are determined by qRT-PCR (a hippocampus tissue of an animal model);

FIG. 2E shows expression levels of miR-25802 in plasma samples of AD patients and healthy volunteers (HAVs) of the same age that are determined by qRT-PCR;

FIG. 2F shows diagnostic and predictive values of miR-25802 in APP/PS1 mice that are analyzed by a receiver operating characteristic (ROC) curve;

FIG. 3A shows levels of pro-inflammatory MI phenotype molecular markers of a resting (inactivated) microglial cell in which miR-25802 is up-regulated/down-regulated that are determined by qRT-PCR;

FIG. 3B shows levels of anti-inflammatory M2 phenotype molecular markers of a resting (inactivated) microglial cell in which miR-25802 is up-regulated/down-regulated that are determined by qRT-PCR;

FIG. 3C shows a level of a pro-inflammatory cytokine TNF-α secreted by a resting (inactivated) microglial cell in which miR-25802 is up-regulated/down-regulated that is determined by ELISA;

FIG. 3D shows a level of a pro-inflammatory cytokine IL-6 secreted by a resting (inactivated) microglial cell in which miR-25802 is up-regulated/down-regulated that is determined by ELISA;

FIG. 3E shows a level of an anti-inflammatory cytokine TGF-β secreted by a resting (inactivated) microglial cell in which miR-25802 is up-regulated/down-regulated that is determined by ELISA;

FIG. 4A shows levels of pro-inflammatory MI phenotype molecular markers of an inflammatory (activated, pro-inflammatory phenotype) microglial cell in which miR-25802 is up-regulated/down-regulated that are determined by qRT-PCR;

FIG. 4B shows levels of pro-inflammatory M2 phenotype molecular markers of an inflammatory (activated, pro-inflammatory phenotype) microglial cell in which miR-25802 is up-regulated/down-regulated that are determined by qRT-PCR;

FIG. 4C shows a level of a pro-inflammatory cytokine TNF-α secreted by an inflammatory (activated, pro-inflammatory phenotype) microglial cell in which miR-25802 is up-regulated/down-regulated that is determined by ELISA;

FIG. 4D shows a level of a pro-inflammatory cytokine IL-6 secreted by an inflammatory (activated, pro-inflammatory phenotype) microglial cell in which miR-25802 is up-regulated/down-regulated that is determined by ELISA;

FIG. 4E shows a level of an anti-inflammatory cytokine TGF-β secreted by an inflammatory (activated, pro-inflammatory phenotype) microglial cell in which miR-25802 is up-regulated/down-regulated that is determined by ELISA;

FIG. 5A shows enrichment analysis results of pathways regulated by miR-25802;

FIG. 5B and FIG. 5C show an expression level of a protein KLF4 in a microglial cell that is determined by Western Blot;

FIG. 6A shows expression levels of NF-κB inflammatory signaling pathway-associated proteins in a resting (inactivated) microglial cell in which miR-25802 is up-regulated/down-regulated that are determined by Western Blot;

FIG. 6B shows relative expression levels of phosphorylated proteins of NF-κB inflammatory signaling pathways p65 and IKKα&β in a resting (inactivated) microglial cell in which miR-25802 is up-regulated/down-regulated that are quantified by Western Blot;

FIG. 6C shows a relative expression level of an NF-κB inflammatory signaling pathway protein IKBα in a resting (inactivated) microglial cell in which miR-25802 is up-regulated/down-regulated that is quantified by Western Blot;

FIG. 6D shows expression levels of NF-κB inflammatory signaling pathway-associated proteins in an inflammatory (activated, pro-inflammatory phenotype) microglial cell in which miR-25802 is up-regulated/down-regulated that are determined by Western Blot;

FIG. 6E shows relative expression levels of phosphorylated proteins of NF-κB inflammatory signaling pathways p65 and IKKα&β in an inflammatory (activated, pro-inflammatory phenotype) microglial cell in which miR-25802 is up-regulated/down-regulated that are quantified by Western Blot; and

FIG. 6F shows a relative expression level of an NF-κB inflammatory signaling pathway protein IKBα in an inflammatory (activated, pro-inflammatory phenotype) microglial cell in which miR-25802 is up-regulated/down-regulated that is quantified by Western Blot.

### DETAILED DESCRIPTION OF THE EMBODIMENTS

The present disclosure provides a miR-25802 cluster as a biomarker for an inflammation-associated disease. The miR-25802 cluster includes miR-25802, and the miR-25802 includes a nucleotide sequence set forth in SEQ ID NO: 2, which is specifically: 5′-UCACGGAUACAGCCUCCUUUGGGA-3′.

The miR-25802 cluster of the present disclosure includes, but is not limited to, miR-25802, and genes with a similar sequence to the miR-25802 all fall within the protection scope of the present disclosure. For example, a derivative obtained by modifying the miR-25802, or a miRNA that has a length of 18 nt to 26 nt and has the same or basically the same function as the miR-25802, or a mRNA that has a length of 18 nt to 26 nt and has the same or basically the same function as the miR-25802, or a derivative obtained by modifying the miRNA that has a length of 18 nt to 26 nt and has the same or basically the same function as the miR-25802 can be used as a biomarker for an inflammation-associated disease. miR-25802 alone cannot be understood as the full protection scope of the present disclosure. The inflammation-associated disease of the present disclosure preferably includes AD.

In the present disclosure, a precursor of the miR-25802 is mir-25802, and the mir-25802 preferably includes a nucleotide sequence set forth in SEQ ID NO: 1, which is specifically:

5′-
UCACGGAUACAGCCUCCUUUGGGAUCCUGCUCUGUUCCCAUGAGACUGU
AUCUGCCUGUGUCCA-3′.

The miR-25802 of the present disclosure is a mature body of mir-25802, and specifically, the miR-25802 is preferably obtained by processing a 5′ arm end of mir-25802. The present disclosure has no strict requirements for a manner of the processing, and a conventional operation can be adopted.

In the present disclosure, with 1, 3, 6, and 9-month-old double-transgenic mice stably transfected with an APP/PSI gene and WT mice as experimental objects, a sequencing technology based on bridging PCR in combination with sequencing by synthesis is used to conduct high-throughput genomic expression-profile next-generation sequencing with a high throughput, a high accuracy, and a low cost. RNA is extracted from a mouse brain tissue by Trizol based method, and then isolated to construct a sequencing gene library, and miR-25802 with a clear characteristic change and a brand-new sequence is discovered. The expression of the miR-25802 is up-regulated in brain tissues of APP/PS1 mice at different ages. In addition, it is determined through reverse transcription and real-time fluorescence quantification detection by qRT-PCR that the expression of miR-25802 in serum of an AD model cell, an AD model animal, and an AD patient is up-regulated, indicating that the miR-25802 cluster is correlated with AD and can be used as a biomarker for diagnosis of AD.

The present disclosure also provides use of the miR-25802 cluster described in the above technical solution in one or more selected from the group consisting of the following a1) to a6):
a1) preparation of a kit for screening an inflammation-associated disease population;
a2) preparation of a kit for diagnosing an inflammation-associated disease population;
a3) preparation of a kit for monitoring a therapeutic status of an inflammation-associated disease population;
a4) preparation of a kit for monitoring prognosis of an inflammation-associated disease population;
a5) preparation of a kit for screening targets related to the inflammation-associated disease; and
a6) preparation of a drug for treating the inflammation-associated disease.

In the present disclosure, the inflammation-associated disease preferably includes AD. The drug of the present disclosure preferably includes one or more selected from the group consisting of the following b1) to b8): b1) a drug for promoting activation of a microglial cell; b2) a drug for promoting a pro-inflammatory cell phenotype of a microglial cell; b3) a drug for promoting release of a pro-inflammatory cytokine; b4) a drug for inhibiting release of an anti-inflammatory cytokine; b5) a drug for promoting an NF-κB signaling pathway in a microglial cell and promoting a neuroinflammatory response; b6) a drug for promoting expression of an MI molecular marker in a microglial cell; b7) a drug for inhibiting expression of an M2 molecular marker in a microglial cell; and b8) a drug for reducing an expression level of KLF4. In the present disclosure, the pro-inflammatory cytokine preferably includes TNF-α and/or IL-6; the anti-inflammatory cytokine preferably includes IL-4 and/or IL-10; the M1 molecular marker in the microglial cell preferably includes iNOS; and the M2 molecular marker in the microglial cell preferably includes ARG1.

The present disclosure takes the miR-25802 cluster as a detection target, and the expression of the miR-25802 cluster in a sample can be measured to screen and diagnose an inflammation-associated disease and monitor a therapeutic status of an inflammation-associated disease population, which enriches the diagnostic markers for AD.

The present disclosure also provides a drug for treating an inflammation-associated disease, where an active ingredient of the drug includes a substance for knocking-out or knocking-down the miR-25802 cluster described in the above technical solution. In the present disclosure, the active ingredient preferably includes one or more selected from the group consisting of a chemical small-molecule drug, a nucleic acid drug, and an antibody drug.

In the present disclosure, the miR-25802 cluster can be knocked out or knocked down to reduce an inflammatory pathological process of AD and effectively prevent and treat inflammation-associated diseases including AD. The present disclosure has no strict requirements for a type of the substance for knocking out or knocking down the miR-25802 cluster, and any substance for knocking out or knocking down the miR-25802 cluster falls within the protection scope of the present disclosure, such as nucleic acid mimics for the miR-25802 cluster, inhibitors for the miR-25802 cluster, nucleic acid drugs, small-molecule compounds, and antibody drugs.

The present disclosure also provides a primer set for detecting the miR-25802 cluster described in the above technical solution, where the primer set includes a reverse transcription primer, an upstream primer, and a downstream primer; and
the reverse transcription primer includes a nucleotide sequence set forth in SEQ ID NO: 3,
the forward primer includes a nucleotide sequence set forth in SEQ ID NO: 4, and
the reverse primer includes a nucleotide sequence set forth in SEQ ID NO: 5.

In the present disclosure, the miR-25802 cluster can be reverse-transcribed with the reverse transcription primer in the primer set and then amplified with the upstream primer and the downstream primer, such that an expression level of miR-25802 can be specifically detected to diagnose AD, predict a risk of developing AD, or predict an outcome of a treatment of AD.

In view of an advantageous role of the primer set of the present disclosure, use of the primer set in preparation of one or more kits selected from the group consisting of the following c1) to c4) also falls within the protection scope of the present disclosure: c1) a kit for screening an inflammation-associated disease population; c2) a kit for diagnosing an inflammation-associated disease population; c3) a kit for monitoring a therapeutic status of an inflammation-associated disease population; and c4) a kit for monitoring prognosis of an inflammation-associated disease population.

The present disclosure also provides a kit for screening an inflammation-associated disease, including the primer set described in the above technical solution. In the present disclosure, the inflammation-associated disease includes AD.

The present disclosure has found that, in addition to being used as a marker for AD, the miR-25802 cluster can be used as a molecular therapeutic target to develop a drug for treating an inflammation-associated disease. Functions of miRNAs of the miR-25802 cluster are subjected to in-depth systematic research. It is found that the miR-25802 cluster can induce the activation of a microglial cell, promote an NF-κB inflammatory signaling pathway, regulate the expression of an inflammation-associated molecular marker in a microglial cell, and promote an inflammatory response, and the down-regulation of expression of miRNAs of the miR-25802 cluster can promote an anti-inflammatory phenotype of a microglial cell and inhibit an inflammatory response. Therefore, the miR-25802 cluster can be knocked out or knocked down to reduce an inflammatory pathological process of AD and effectively prevent and treat AD. The present disclosure discovers a relationship between the miR-25802 cluster and AD, and provides a potential new target to exert an anti-inflammatory effect, which solves the problem that there is a lack of diagnostic markers for AD at a gene level in the prior art, and is conducive to solving the current situation that there is a lack of effective targets for a treatment of inflammation including AD in the prior art.

In order to further illustrate the present disclosure, the technical solutions of the miR-25802 cluster as a biomarker for AD and the use of the miR-25802 cluster provided in the present disclosure are described in detail below in connection with drawings and Examples, but these Examples should not be understood as limiting the claimed scope of the present disclosure.

In the specific Examples of the present disclosure, unless otherwise specified, steps involved all are routine steps, and reagents used can be conventionally purchased or prepared according to product instructions.

### Example 1

Detection of Differentially-Expressed miRNAs in a Pathological Process of AD Through miRNA High-Throughput Sequencing

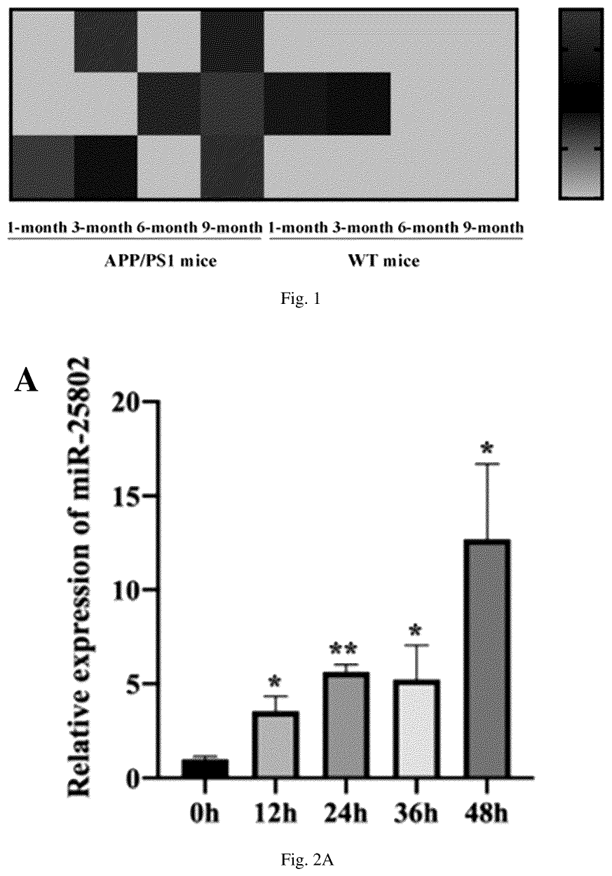

APP/PS1 mice (which were denoted as APP/PS1 mice and purchased from Zhishan (Beijing) Health Medical Research Institute) and Wide type mice (which were denoted as WT mice and purchased from Zhishan (Beijing) Health Medical Research Institute) were adopted as experimental materials, and the mice were sacrificed through excessive inhalation of diethyl ether. Brain tissues of 1, 3, 6, and 9 month-old APP/PSI mice and WT mice each were collected, and a cerebral cortex and a hippocampus were separated from each brain tissue, then immediately placed in liquid nitrogen overnight, and then stored in a −80° C. refrigerator. A sequencing technology based on bridging PCR in combination with sequencing by synthesis was used to conduct high-throughput genomic expression-profile next-generation sequencing with a high throughput, a high accuracy, and a low cost. Total RNA was extracted from a mouse cortex and hippocampus by Trizol based method and then isolated to construct a sequencing gene library. The sequencing gene library was subjected to single-end sequencing with Illumina HiSeq 2500. Quality of raw sequencing data was evaluated by FastQC, and miRDeep2 software was used to align miRNA with a reference genome, analyze a secondary structure of miRNA, and analyze differential expression of miRNA. Experimental results showed that there was a non-coding RNA differentially expressed in hippocampi and cortices of APP/PS1 mice and WT mice of the same age (as shown in FIG. 1, results were expressed as mean±SEM (n=3)). The non-coding RNA was denoted as miR-25802, and a nucleotide sequence of the non-coding RNA was set forth in SEQ ID NO: 2, which was specifically: 5′-UCACGGAUACAGCCUCCUUUGGGA-3′. The miR-25802 was a mature body, and a nucleotide sequence of mir-25802 as a precursor of miR-25802 was set forth in SEQ ID NO: 1, which was specifically: 5′-UCACGGAUACAGCCUCCUUUGGGAUCCUGCUCUGUUCCCAUGAGACUGUAUCUGCCU GUGUCCA-3′. A sequence of a reverse transcription primer for the miR-25802 was set forth in SEQ ID NO: 3, and was specifically: 5′-GTCGTATCCAGTGCAGGGTCCGAGGTATTCGCACTGGATACGACTCCCAA-3′; a sequence of a forward primer for quantitative PCR (qPCR) detection of the miR-25802 was set forth in SEQ ID NO: 4, and was specifically: 5′-CGTCACGGATACAGCCTCCT-3′; and a sequence of a reverse primer for qPCR detection of the miR-25802 was set forth in SEQ ID NO: 5, and was specifically: 5′-AGTGCAGGGTCCGAGGTATT-3′. The above steps were entrusted to Sangon Biotech (Shanghai) Co., Ltd.

### Example 2

a Change of Expression of miRNAs of a miR-25802 Cluster in an AD Model Cell
(1) Monoclonal strains were acquired through cell cultivation, liposome-based transient transfection, antibiotic-based pressurized screening, and limited dilution, and related proteins were detected by Western Blot or ELISA, such as to construct human neuroblastoma cells (APPswe cells) stably transfected with a human-mouse chimeric APP gene, which was specifically described in the literature (Wang, C. Y., et al. (2011). Huperzine A activates Wnt/β-catenin signaling and enhances the nonamyloidogenic pathway in an Alzheimer transgenic mouse model. Neuropsychopharmacology. 36 (5), 1073-1089.).
(2) The APPswe cells obtained in the step (1) were cultivated in Dulbecco's Modified Eagle Medium (DMEM) with 10% of fetal bovine serum (FBS) at 5% CO₂ and 37° C. 1 μg/mL puromycin solution was used to maintain phenotypic traits of cells of stably-transfected strains. When a cell confluency was 80%, a 300 μM copper ion solution was used to treat cells. After the APPswe cells were conducted induction treatment with copper ions, the copper ions formed chelates with APP and AB, which aggravated the production and deposition of AB, and induced an oxidative stress and the apoptosis of nerve cells. Therefore, APPswe cells treated with copper ions could be used to simulate a pathological state of AD nerve cells and investigate a drug-acting mechanism.

APPswe cells were randomly collected at different time points after the copper ion treatment, and subjected to total RNA extraction by a Trizol based method (a biological kit of CoWin Biotech, CW0581). Then, a reverse transcription reaction was conducted by a stem-loop method (Nanjing Vazyme, MIR-101), and an expression level of miR-25802 in the APPswe cells was quantitatively detected by qRT-PCR (Nanjing Vazyme, MQ-101), where specific operations were conducted according to instructions of reagent. A sequence of a reverse transcription primer was set forth in SEQ ID NO: 3, a sequence of a forward primer for real-time fluorescence quantification detection was set forth in SEQ ID NO: 4, and a sequence of a reverse primer for real-time fluorescence quantification detection was set forth in SEQ ID NO: 5.

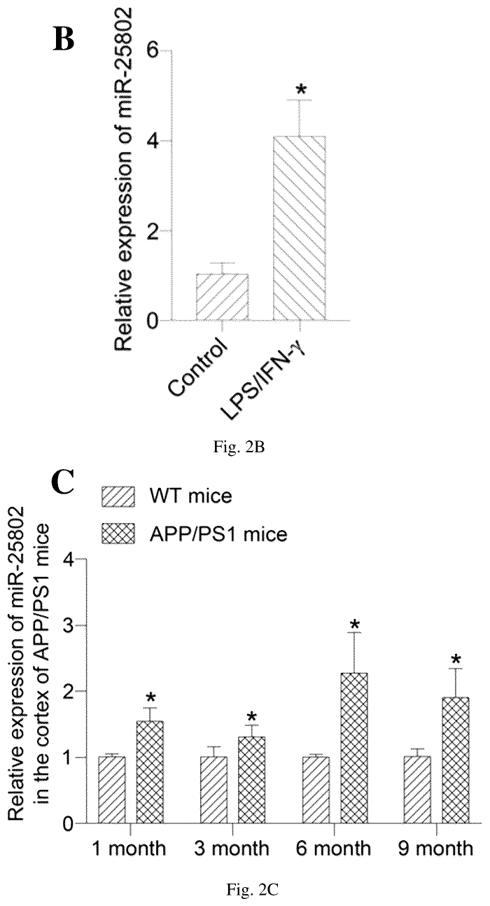

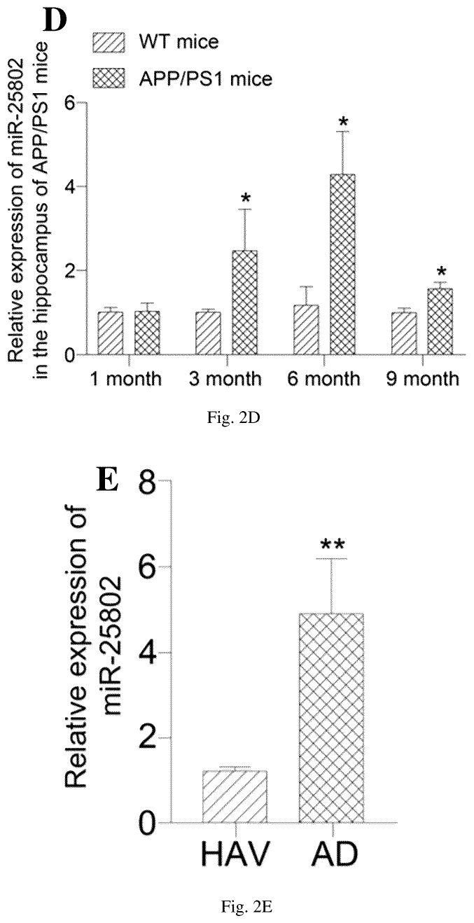

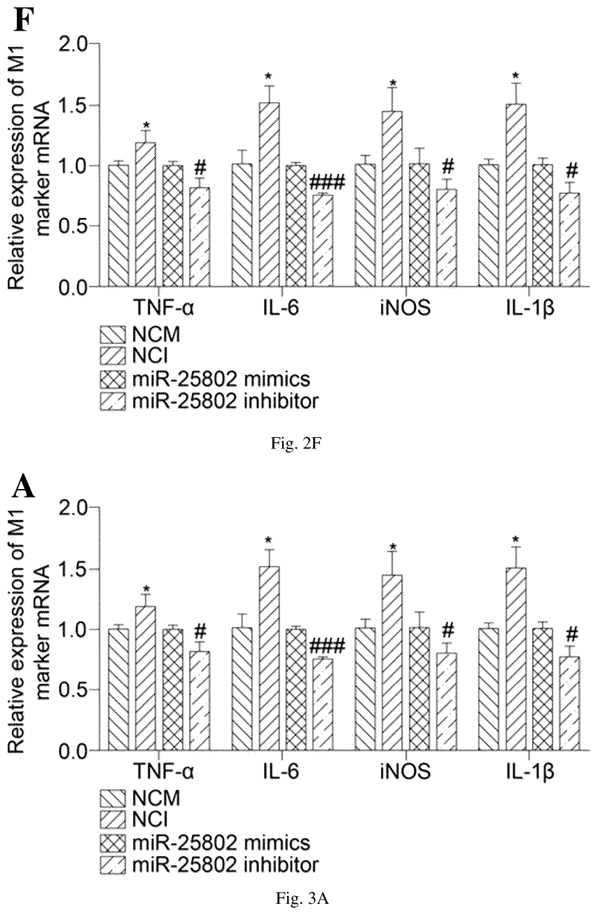

Test results are shown in FIG. 2A, where the results are expressed as mean±SEM (n=3), * represents P<0.05 compared with a result at 0 h when no copper ion is added, and ** represents P<0.01 compared with the result at 0 h.

It can be seen from FIG. 2A that a cell damage induced by copper ions stimulation increases over time, and an expression level of miR-25802 increases accordingly, indicating that the expression of miR-25802 is up-regulated during a pathological process of AD.
(3) Mouse microglial cells EOC20 were purchased from American Type Culture Collection (ATCC), and cultivated in DMEM including 10% of FBS and 20% of a LADMAC conditioned medium at 5% CO₂ and 37° C. The EOC20 cells were inoculated into a six-well plate at 1×10⁵ cells/mL, and lipopolysaccharide (LPS) at a final concentration of 100 ng/ml and IFN-γ at a final concentration of 1 ng/ml were added to each well; 24 h later, total RNA was extracted by a Trizol based method (a biological kit of CoWin Biotech, CW0581), reverse-transcribed (Nanjing Vazyme, R323), and amplified by qRT-PCR (Nanjing Vazyme, Q711); and with a Actb gene as an internal reference gene, relative expression levels of mRNAs for pro-inflammatory molecules TNF-α and IL-6 were determined. In this model, the expression levels of the pro-inflammatory molecules were significantly up-regulated, indicating that LPS/IFN-γ co-induced the activation of the mouse microglial cells EOC20 to present the MI pro-inflammatory cell phenotype, and thus an in vitro inflammatory cell model was successfully constructed, which was denoted as LPS/IFN-γ. A model without LPS/IFN-γ induction was adopted as a control group, which was referred to as contro.

An expression level of miR-25802 in the neuroinflammatory cell model was detected through reverse transcription and real-time fluorescence quantification detection by qRT-PCR, where a sequence of a reverse transcription primer was set forth in SEQ ID NO: 3, a sequence of a forward primer for real-time fluorescence quantification detection was set forth in SEQ ID NO: 4, and a sequence of a reverse primer for real-time fluorescence quantification detection was set forth in SEQ ID NO: 5.

Test results are shown in FIG. 2B, where the results are expressed as mean±SEM (n=3), and * represents P<0.05 compared with the control group.

It can be seen from FIG. 2B that the expression level of miR-25802 in the inflammatory cell model significantly increases.

### Example 3

A Change of Expression of miRNAs of a miR-25802 Cluster in an AD Model Animal

1, 3, 6, and 9 month-old APP/PS1 double-transgenic mice (which were denoted as APP/PS1 mice and were purchased from Zhishan (Beijing) Health Medical Research Institute) were adopted as experimental groups, and 1, 3, 6, and 9 month-old Wide type control mice were adopted as control groups (which were denoted as WT mice and were purchased from Zhishan (Beijing) Health Medical Research Institute). The mice were sacrificed by anesthesia. And cortex and hippocampus tissues were rapidly isolated from each of the 1, 3, 6, and 9 month-old APP/PS1 double-transgenic mice and WT control mice on ice and then frozen in liquid nitrogen. Total miRNA was extracted from each of the mouse cortex and hippocampus tissues by a Trizol based method. The concentration and purity of the total RNA were determined by ultraviolet spectrophotometry, and a change of expression of miR-25802 during a pathological process of AD was detected by qRT-PCR. Test results are shown in FIG. 2C and FIG. 2D, where FIG. 2C shows detection results of mouse cortex tissues, and FIG. 2D shows detection results of mouse hippocampus tissues. The results of C and D are expressed as mean±SEM (n=3); and * represents P<0.05 of APP/PS1 mice compared with WT mice.

It can be seen from 2C and FIG. 2D that expression levels of miR-25802 in cortex and hippocampus tissues of the animal model significantly increase compared with control mice (WT mice) at ages of 1, 3, 6, and 9 months.

### Example 4

A Change of Expression of miRNAs of a miR-25802 Cluster in Serum of an AD Patient

Serum was collected from each of 11 AD patients and 11 Healthy age-matched volunteers (HAVs) with the same age to obtain experimental materials, total RNA was extracted from patients and HAVs. The concentration and purity of RNA were determined by ultraviolet spectrophotometry, and a content of miR-25802 in serum of each AD patient was detected by qRT-PCR. An ROC curve was used to analyze the ability of differentially-expressed miR-25802 as a diagnostic indicator to distinguish between AD patients and healthy volunteers. Analysis results are shown in FIG. 2E and FIG. 2F. FIG. 2E shows detection results of qRT-PCR, where the results are expressed as mean±SEM (n=11), ** represents P<0.01 of AD patients compared with HAV. FIG. 2F shows analysis results of the ROC curve, where an area under the curve (AUC) of the ROC curve is 0.920 (CI: 0.800-1.00, P<0.01), a sensitivity is 87.5%, and a specificity is 81.8%.

It can be seen from FIG. 2E and FIG. 2F that a relative expression level of miR-25802 in blood of the AD patients significantly increases, the ROC curve detection has a high sensitivity and specificity, and the use of differential relative expression of miR-25802 as a diagnostic method can effectively distinguish patients from HAVs, with a high accuracy.

### Example 5

An Impact of Dysregulated Expression of miRNAs of a miR-25802 Cluster on a Phenotype and an Inflammatory Response of a Resting Microglial Cell
(1) Based on a miRNA mimic and a miRNA inhibitor, a liposome-based transient transfection technology was used to construct a cell model in which a miRNA was over-expressed or knocked out, which was specifically as follows:
mouse microglial cells EOC20 were equally divided into four groups, which were denoted as an NCM group, an NCI group, a miR-25802 mimic group, and a miR-25802 inhibitor group, respectively;
in the NCM group, a 50 nM miRNA unrelated sequence negative control (negative control, NCM: SEQ ID NO: 6: 5′-UUGUACUACACAAAAGUACUG-3′) was transiently transfected by a liposome;
in the NCI group, a 50 nM miRNA unrelated sequence negative control (negative control, NCI: SEQ ID NO: 7: 5′-CAGUACUUUUGUGUAGUACAA-3′) was transiently transfected by a liposome;
in the miR-25802 mimic group, a 50 nM novel miR-25802 mimic (SEQ ID NO: 2: 5′-UCACGGAUACAGCCUCCUUUGGGA-3′) was transiently transfected by a liposome;
in the miR-25802 inhibitor group, a 50 nM novel miR-25802 inhibitor (SEQ ID NO: 8: 5′-UCCCAAAGGAGGCUGUAUCCGUGA-3′) was transiently transfected by a liposome.

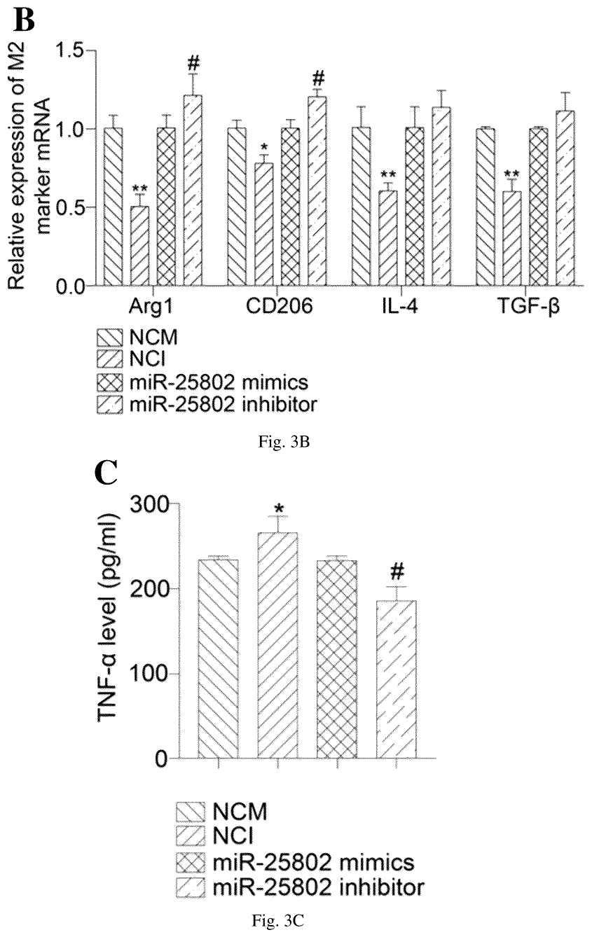

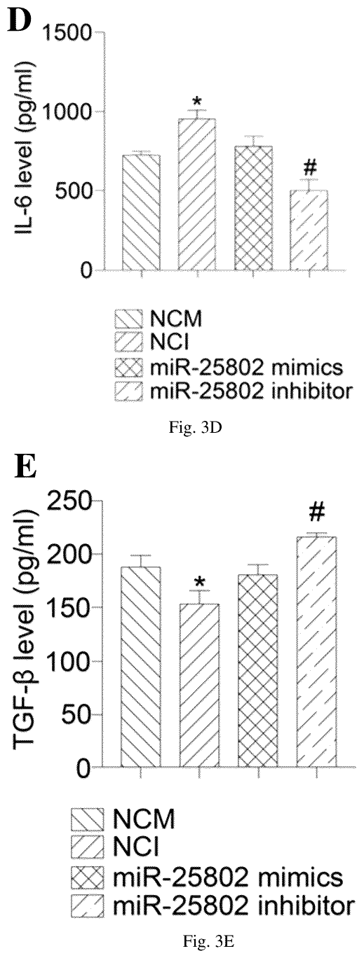

Treated cells in each treatment group were incubated at 37° C. At 24 h of the incubation, a miRNA expression level was detected, and at 48 h of the incubation, secretory cytokines were detected.
(2) After the step (1) was completed, qRT-PCR and ELISA were used to detect inflammation-associated phenotype markers in microglial cells. Detection results are shown in FIG. 3A to FIG. 3E. FIG. 3A shows levels of pro-inflammatory M1 phenotype molecular markers of the resting (inactivated) microglial cell in which miR-25802 is up-regulated/down-regulated that are determined by qRT-PCR; FIG. 3B shows levels of anti-inflammatory M2 phenotype molecular markers of the resting (inactivated) microglial cell in which miR-25802 is up-regulated/down-regulated that are determined by qRT-PCR; FIG. 3C shows a level of a pro-inflammatory cytokine TNF-α secreted by the resting (inactivated) microglial cell in which miR-25802 is up-regulated/down-regulated that is determined by ELISA; FIG. 3D shows a level of a pro-inflammatory cytokine IL-6 secreted by a resting (inactivated) microglial cell in which miR-25802 is up-regulated/down-regulated that is determined by ELISA; and FIG. 3E shows a level of an anti-inflammatory cytokine TGF-β secreted by a resting (inactivated) microglial cell in which miR-25802 is up-regulated/down-regulated that is determined by ELISA. In FIG. 3A to FIG. 3E, the results are expressed as mean±SEM (n=4); * represents P<0.05 compared with NCM; * represents P<0.01 compared with NCM; #represents P<0.05 compared with NCI; and ###represents P<0.001 compared with NCI.

It can be seen from FIG. 3A to FIG. 3E that the overexpression of miR-25802 can induce the activation and pro-inflammatory phenotype transformation of the resting microglial cell, and promote the secretion of pro-inflammatory cytokines.

### Example 6

An Impact of Dysregulated Expression of miRNAs of a miR-25802 Cluster on a Phenotype and an Inflammatory Response of an Inflammatory Microglial Cell

According to the step (3) of Example 2, inflammatory cell models were constructed. An expression level of miR-25802 in an inflammatory cell model was up-regulated or down-regulated by cotransfection of a miR-25802 mimic or a miR-25802 inhibitor with a liposome, which was specifically as follows:
the inflammatory cell models were equally divided into four groups, which were denoted as an NCM group, an NCI group, a miR-25802 mimic group, and a miR-25802 inhibitor group, respectively;
in the NCM group, a 50 nM miRNA unrelated sequence negative control (negative control, NCM: SEQ ID NO: 6: 5′-UUGUACUACACAAAAGUACUG-3′) was transiently transfected by a liposome;
in the NCI group, a 50 nM miRNA unrelated sequence negative control (negative control, NCI: SEQ ID NO: 7: 5′-CAGUACUUUUGUGUAGUACAA-3′) was transiently transfected by a liposome;
in the miR-25802 mimic group, a 50 nM novel miR-25802 mimic (SEQ ID NO: 2: 5′-UCACGGAUACAGCCUCCUUUGGGA-3′) was transiently transfected by a liposome;
in the miR-25802 inhibitor group, a 50 nM novel miR-25802 inhibitor (SEQ ID NO: 8: 5′-UCCCAAAGGAGGCUGUAUCCGUGA-3′) was transiently transfected by a liposome.

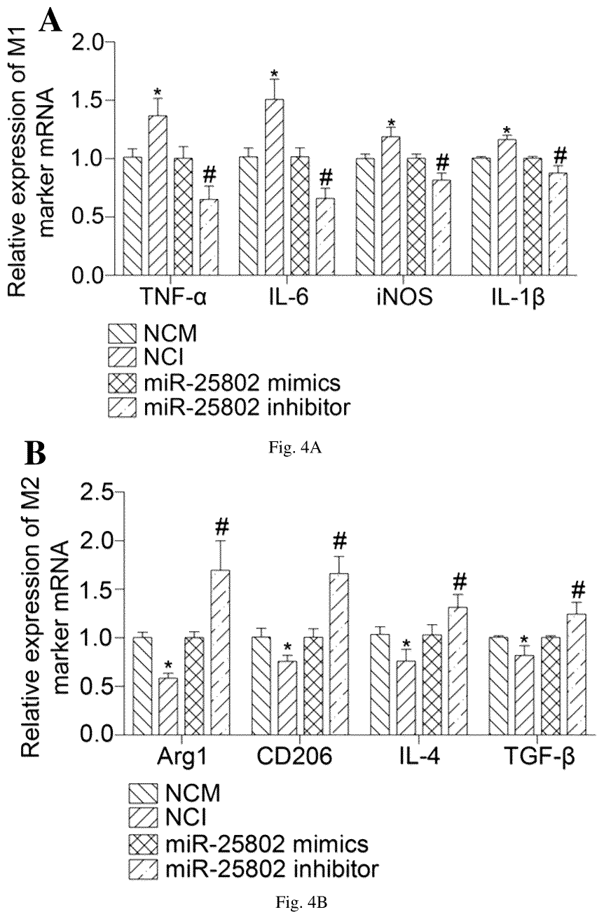

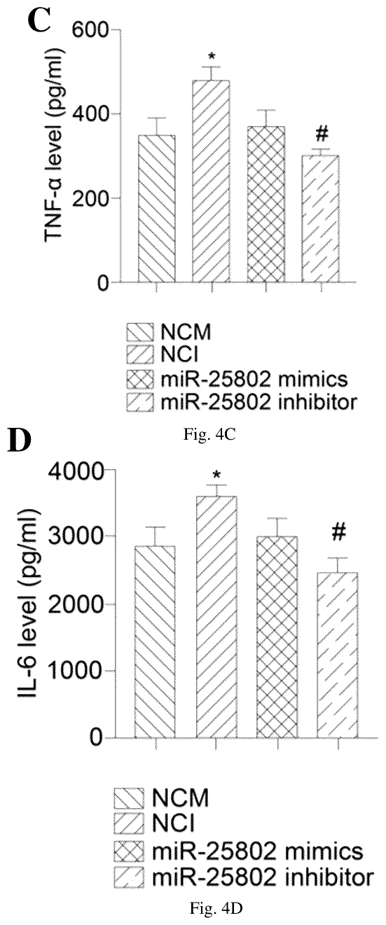

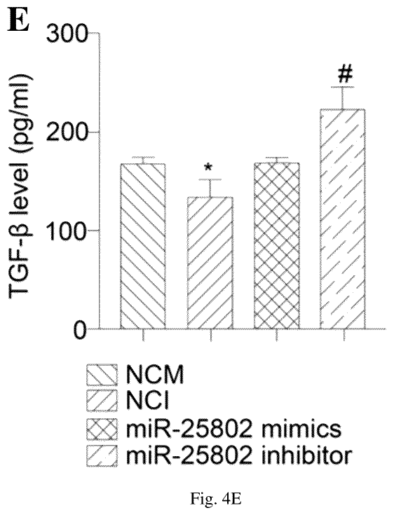

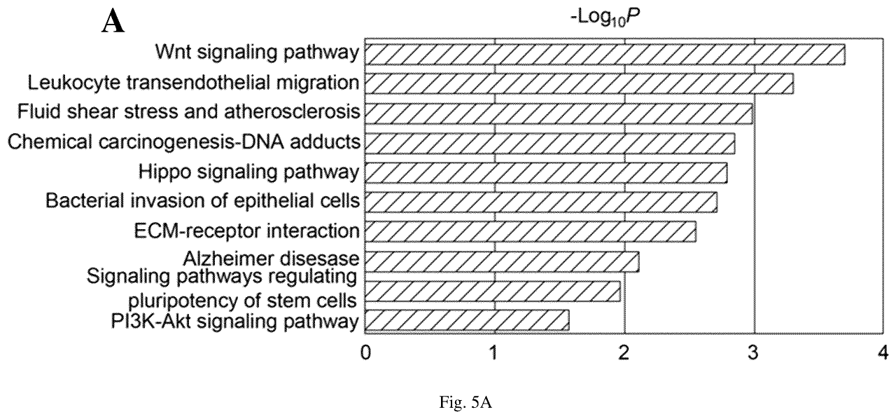

24 h or 48 h after transfection, qRT-PCR and ELISA were used for detection, respectively. Detection results are shown in FIG. 4A to FIG. 4E. FIG. 4A shows levels of pro-inflammatory M1 phenotype molecular markers of the inflammatory (activated, pro-inflammatory phenotype) microglial cell in which miR-25802 is up-regulated/down-regulated that are determined by qRT-PCR; FIG. 4B shows levels of pro-inflammatory M2 phenotype molecular markers of the inflammatory (activated, pro-inflammatory phenotype) microglial cell in which miR-25802 is up-regulated/down-regulated that are determined by qRT-PCR; FIG. 4C shows a level of a pro-inflammatory cytokine TNF-α secreted by the inflammatory (activated, pro-inflammatory phenotype) microglial cell in which miR-25802 is up-regulated/down-regulated that is determined by ELISA; FIG. 4D shows a level of a pro-inflammatory cytokine IL-6 secreted by an inflammatory (activated, pro-inflammatory phenotype) microglial cell in which miR-25802 is up-regulated/down-regulated that is determined by ELISA; and FIG. 4E shows a level of an anti-inflammatory cytokine TGF-β secreted by an inflammatory (activated, pro-inflammatory phenotype) microglial cell in which miR-25802 is up-regulated/down-regulated that is determined by ELISA. In FIG. 4A to FIG. 4E, the results are expressed as mean±SEM (n=4); * represents P<0.05 compared with NCM; and #represents P<0.05 compared with NCI.

It can be seen from FIG. 4A to FIG. 4E that the down-regulation of expression of miR-25802 can promote the anti-inflammatory phenotype transformation of microglial cells, inhibit the release of pro-inflammatory cytokines, promote the expression of anti-inflammatory molecular markers, and alleviate inflammatory responses.

### Example 7

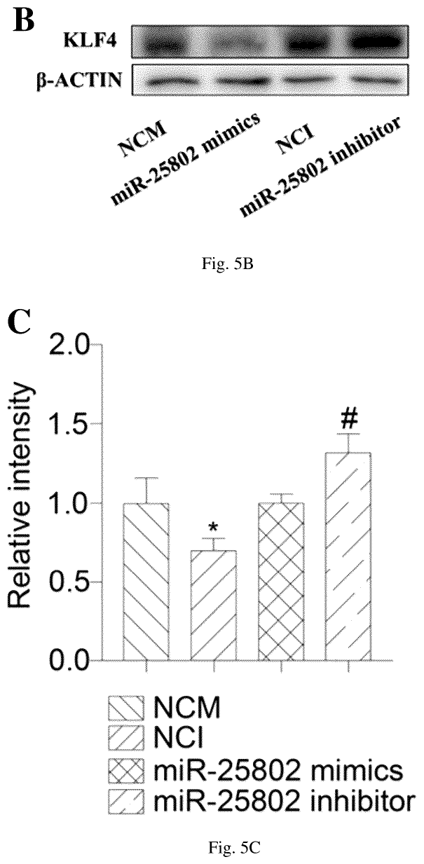

Prediction of miRNA Target Genes of a miR-25802 Cluster
(1) Potential binding targets of miR-25802 were predicted by bioinformatics software miRDB and miRanda, and the binding genes predicted for miR-25802 were subjected to KEGG pathway enrichment analysis by Metascape on-line software. Analysis results are shown in FIG. 5A. It can be seen from FIG. 5A that the target genes of miR-25802 are enriched in pathways related to inflammation such as AD and immune responses.

### Example 8

Specific Regulation of Expression of KLF4 by miRNAs of a miR-25802 Cluster at a Translational Level

A microglial cell model in which miR-25802 was over-expressed or knocked down was constructed using liposome-based transient transfection technology, based on a miRNA mimic/inhibitor.

Specifically, microglial cells were equally divided into four groups, which were denoted as an NCM group, an NCI group, a miR-25802 mimic group, and a miR-25802 inhibitor group, respectively;
in the NCM group, a 50 nM miRNA unrelated sequence negative control (negative control, NCM: SEQ ID NO: 6: 5′-UUGUACUACACAAAAGUACUG-3′) was transiently transfected by a liposome;
in the NCI group, a 50 nM miRNA unrelated sequence negative control (negative control, NCI: SEQ ID NO: 7, 5′-CAGUACUUUUGUGUAGUACAA-3′) was transiently transfected by a liposome;
in the miR-25802 mimic group, a 50 nM novel miR-25802 mimic (SEQ ID NO: 2: 5′-UCACGGAUACAGCCUCCUUUGGGA-3′) was transiently transfected by a liposome;
in the miR-25802 inhibitor group, a 50 nM novel miR-25802 inhibitor (SEQ ID NO: 8: 5′-UCCCAAAGGAGGCUGUAUCCGUGA-3′) was transiently transfected by a liposome.

48 h after transfection, a total protein was extracted from the cells through RIPA lysis and ultrasonic extraction, and a visible light absorbance was determined by BCA assay for quantification. A protein was reduced by dithiothreitol and boiled for denaturation, and an expression level of the KLF4 protein in cells was detected by Western Blot. Results are shown in FIG. 5B and FIG. 5C, where the results are expressed as mean±SEM (n=4); * represents P<0.05 compared with NCM; and #represents P<0.05 compared with NCI.

It can be seen from FIG. 5B and FIG. 5C that the up-regulation of expression of miR-25802 can negatively regulate the expression level of the specific target KLF4 at a translational level and reduce expression level of the protein KLF4, while the down-regulation of expression level of miR-25802 can promote the high expression of KLF4.

### Example 9

Regulation of NF-κB Inflammatory Signaling Pathways by miRNAs of a miR-25802 Cluster

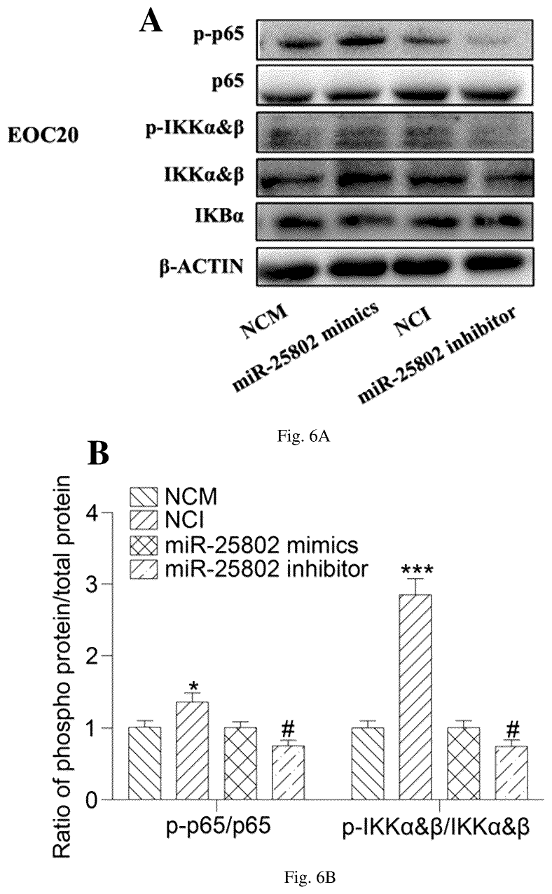

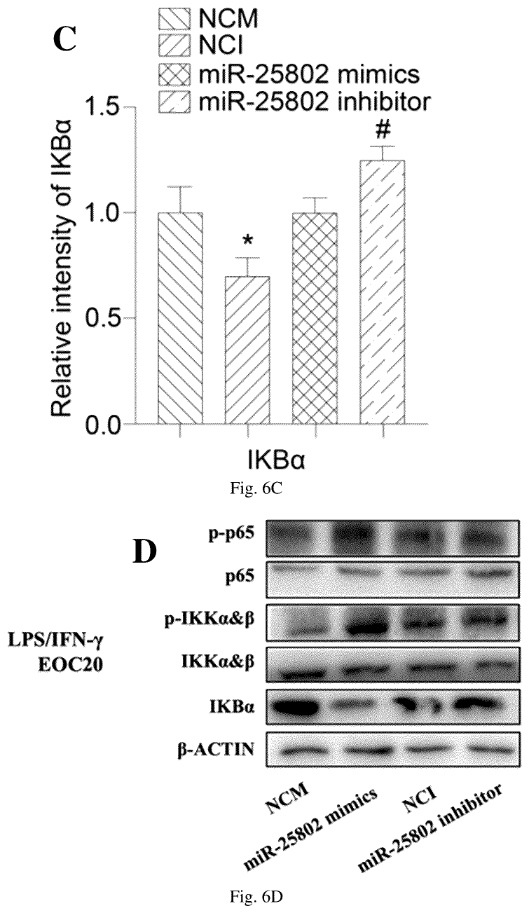

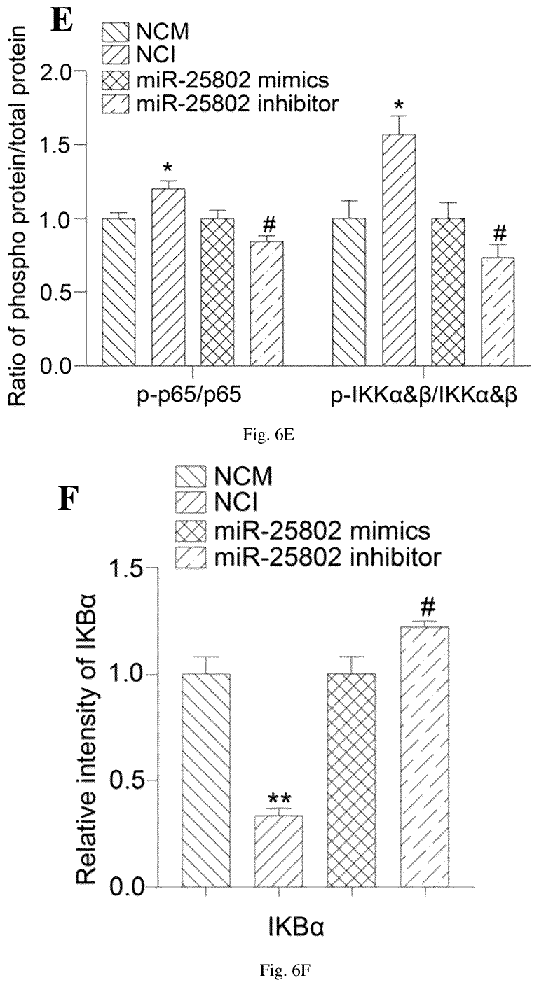

According to the steps of Example 6, an inflammatory cell model in which miR-25802 was over-expressed or knocked down was constructed using a liposome-based transfection technology. Expression levels of NF-κB signaling pathway-associated molecules were detected by Western Blot. Results are shown in FIG. 6A to FIG. 6F. FIG. 6A shows expression levels of NF-κB inflammatory signaling pathway-associated proteins in the resting (inactivated) microglial cell in which miR-25802 is up-regulated/down-regulated that are determined by Western Blot; FIG. 6B shows relative expression levels of phosphorylated proteins of NF-κB inflammatory signaling pathways p65 and IKKα&β in the resting (inactivated) microglial cell in which miR-25802 is up-regulated/down-regulated that are quantified by Western Blot; FIG. 6C shows a relative expression level of an NF-κB inflammatory signaling pathway protein IKBα in the resting (inactivated) microglial cell in which miR-25802 is up-regulated/down-regulated that is quantified by Western Blot; FIG. 6D shows expression levels of NF-κB inflammatory signaling pathway-associated proteins in the inflammatory (activated, pro-inflammatory phenotype) microglial cell in which miR-25802 is up-regulated/down-regulated that are determined by Western Blot; FIG. 6E shows relative expression levels of phosphorylated proteins of NF-κB inflammatory signaling pathways p65 and IKKα&β in an inflammatory (activated, pro-inflammatory phenotype) microglial cell in which miR-25802 is up-regulated/down-regulated that are quantified by Western Blot; and FIG. 6F shows a relative expression level of an NF-κB inflammatory signaling pathway protein IKBα in an inflammatory (activated, pro-inflammatory phenotype) microglial cell in which miR-25802 is up-regulated/down-regulated that is quantified by Western Blot. In FIG. 6A to FIG. 6F, the results are expressed as mean±SEM (n=4); * represents P<0.05 compared with NCM; ** represents P<0.01 compared with NCM; *** represents P<0.001 compared with NCM; and #represents P<0.05 compared with NCI.

It can be seen from FIG. 6A to FIG. 6F that the overexpression of miR-25802 can up-regulate expression levels of NF-κB signaling pathway-associated molecules in resting microglial cells, and the inhibition of miR-25802 can down-regulate expression levels of NF-κB signaling pathway-associated molecules in microglial cells with a pro-inflammatory phenotype.

According to the above examples, an expression level of the miR-25802 provided by the present disclosure significantly increases during a pathological process of AD, and an ROC curve based on expression levels in serum shows that the miR-25802 has an excellent diagnostic effect, can be used as a biomarker for detection of AD, and can negatively regulate an expression level of the gene KLF4. The overexpression of miR-25802 can induce and promote pro-inflammatory phenotype transformation of microglial cells, up-regulate levels of inflammatory cytokines, and promote inflammatory responses. The overexpression of miR-25802 can up-regulate an activity of an NF-κB inflammatory signaling pathway, while the knock down of miR-25802 can down-regulate an activity of an NF-κB inflammatory signaling pathway, promote inflammatory phenotype transformation of microglial cells, and effectively prevent and treat AD.

Although the present disclosure has been described in detail through the above Examples, the Examples are merely some rather than all of the examples of the present disclosure. All other examples obtained by a person based on these Examples without creative efforts shall fall within the protection scope of the present disclosure.

1-11. (canceled)

12. A miR-25802 cluster as a biomarker for an inflammation-associated disease, wherein the miR-25802 cluster comprises:
miR-25802, wherein the miR-25802 comprises a nucleotide sequence set forth in SEQ ID NO: 2.

13. The miR-25802 cluster according to claim 12, wherein a precursor of the miR-25802 is mir-25802, and the mir-25802 comprises a nucleotide sequence set forth in SEQ ID NO: 1.

14. A method for preparing a drug for treating the inflammation-associated disease, comprising using the miR-25802 cluster according to claim 12.

15. The method according to claim 14, wherein the inflammation-associated disease comprises Alzheimer's disease (AD).

16. The method according to claim 14, wherein the drug comprises one or more selected from the group consisting of the following b1) to b8):
b1) a drug for promoting activation of a microglial cell;
b2) a drug for promoting a pro-inflammatory cell phenotype of a microglial cell;
b3) a drug for promoting release of a pro-inflammatory cytokine, wherein the pro-inflammatory cytokine comprises TNF-α and/or IL-6;
b4) a drug for inhibiting release of an anti-inflammatory cytokine, wherein the anti-inflammatory cytokine comprises IL-4 and/or IL-10;
b5) a drug for promoting an NF-κB signaling pathway in a microglial cell and promoting a neuroinflammatory response;
b6) a drug for promoting expression of an M1 molecular marker in a microglial cell, wherein the M1 molecular marker in the microglial cell comprises iNOS;
b7) a drug for inhibiting expression of an M2 molecular marker in a microglial cell, wherein the M2 molecular marker in the microglial cell comprises ARG1; and
b8) a drug for reducing an expression level of KLF4.

17. A drug for treating an inflammation-associated disease, wherein an active ingredient of the drug comprises a substance for knocking-out or knocking-down the miR-25802 cluster according to claim 12.

18. The drug according to claim 17, wherein the active ingredient comprises a chemical small-molecule drug, a nucleic acid drug, and an antibody drug.

19. A primer set for detecting the miR-25802 cluster according to claim 12, wherein the primer set comprises a reverse transcription primer, an upstream primer, and a downstream primer; and
the reverse transcription primer comprises a nucleotide sequence set forth in SEQ ID NO: 3,
the upstream primer comprises a nucleotide sequence set forth in SEQ ID NO: 4, and
the downstream primer comprises a nucleotide sequence set forth in SEQ ID NO: 5.

20. A kit for screening an inflammation-associated disease, comprising the primer set according to claim 19.

21. A method for knocking-out or knocking-down the miR-25802 cluster according to claim 12, comprising: using a liposome for transient transfection of a substance for knocking-out or knocking-down the miR-25802 cluster.

22. A method for preparing a kit for screening an inflammation-associated disease population, comprising using the miR-25802 cluster according to claim 12.

23. A method for preparing a kit for diagnosing an inflammation-associated disease population, comprising using the miR-25802 cluster according to claim 12.

24. A method for preparing a kit for monitoring a therapeutic status of an inflammation-associated disease population, comprising using the miR-25802 cluster according to claim 12.

25. A method for preparing a kit for monitoring prognosis of an inflammation-associated disease population, comprising using the miR-25802 cluster according to claim 12.

26. A method for preparing a kit for screening targets related to the inflammation-associated disease, comprising using the miR-25802 cluster according to claim 12.
